(19) United States
(12) Patent Application Publication
(10) Pub. No.: US 2013/0336453 A1
(43) Pub. Date: Dec. 19, 2013

(54) DIGITAL X-RAY DETECTOR, HOUSING AND X-RAY PHOTOGRAPHING APPARATUS USING THE SAME

(71) Applicant: Rayence Co., Ltd., Gyeonggi-do (KR)

(72) Inventors: Se Hee PARK, Gyeonggi-do (KR); Sang Jo Park, Gyeonggi-do (KR); Yeong Kyun Kim, Gyeonggi-do (KR); Sang Jin Park, Gyeonggi-do (KR)

(73) Assignee: Rayence Co., Ltd., Gyeonggi-do (KR)

(21) Appl. No.: 13/971,739

(22) Filed: Aug. 20, 2013

Related U.S. Application Data
(63) Continuation of application No. 13/160,962, filed on Jun. 15, 2011, now abandoned.

(30) Foreign Application Priority Data
Jun. 15, 2010 (KR) 10-2010-0056543

(51) Int. Cl. A61B 6/00

(52) U.S. Cl. CPC A61B 6/4429
USPC 378/62; 378/189

(57) ABSTRACT

Provided are a table type digital X-ray detector, which is capable of implementation within a digital X-ray photographing apparatus through easy installation without replacing an existing X-ray photographing apparatus, a housing for the table type digital X-ray detector, and an X-ray photographing apparatus using the table type digital X-ray detector. The X-ray photographing apparatus may include an X-ray source: a support provided at a position facing the X-ray source; a housing configured to be fixedly attachable to the support, at least a portion of the top surface of the housing being made of an X-ray transmittable material; and a digital X-ray sensing module embedded in the housing.

### CROSS REFERENCE TO PRIOR APPLICATIONS

The present application claims priority under 35 U.S.C. §119 to Korean Patent Application No. 10-2010-0056543 (filed on Jun. 15, 2010), which is hereby incorporated by reference in its entirety.

### FIELD OF INVENTION

Apparatuses and methods consistent with the present invention relate to a table type digital X-ray detector, a housing for the same, and an X-ray photographing apparatus using the same, and more particularly, to a table type digital X-ray detector, which is capable of implementing a digital X-ray photographing apparatus through easy installation without replacing an existing X-ray photographing apparatus, a housing for the same, and an X-ray photographing apparatus using the same.

### BACKGROUND OF THE INVENTION

An X-ray photographing apparatus refers to an apparatus that diagnoses a patient or animal's health condition by irradiating an X-ray on a subject to be examined, such as a patient or animal, and acquiring an X-ray image of the subject from the X-ray transmitted through the patient or animal's body.

A related art X-ray photographing apparatus includes an X-ray source for generating an X-ray and irradiating the generated X-ray on a subject to be examined, and an image detector for acquiring an X-ray image of the subject by detecting the X-ray transmitted through the subject. Examples of the image detector include a film type X-ray sensing module such as an X-ray film, and a digital X-ray sensing module using an image sensor array.

The film type X-ray sensing module is time-consuming to use to form an image and also requires a space for storing a film. For these reasons. digital sensing modules using semiconductor image sensors, such as thin film transistor (TFT) images, charge coupled device (CCD) images, complementary metal oxide semiconductor (CMOS) image sensors, etc., have recently been developed and are now on sale.

A digital X-ray sensing module is a device that converts an X-ray image to a digital signal and may be roughly classified into a direct X-ray sensing module and an indirect X-ray sensing module.

The direct X-ray sensing module directly converts an X-ray to electric charges using an optical conductive film, such as amorphous selenium (Se). Meanwhile, the indirect X-ray sensing module converts an X-ray to visible light using a phosphor panel (scintillator panel) and converts the visible light to electric charges using a photoelectric transformation element.

The direct X-ray sensing module is superior in resolution. However, the direct X-ray sensing module is vulnerable to dielectric breakdown because it uses a high voltage, leading to degradation in the reliability thereof. In addition, the direct X-ray sensing module cannot easily use a photoconductive material having low dark current, high sensitivity, and thermal stability.

The indirect X-ray sensing module uses a photodiode to generate signal charges. Since the indirect X-ray sensing module does not use a high voltage as opposed to the direct X-ray sensing module, dielectric breakdown does not occur. In addition, since technology for phosphor materials and photodiodes has already reached a significant level, it is easy to manufacture the X-ray sensing module. Therefore, much attention has recently been paid to the indirect X-ray sensing module. The indirect X-ray sensing module commonly uses a scintillator panel and a TFT panel for the purpose of realizing a large-size module. That is, a related art digital X-ray sensing module includes a scintillator panel, which is configured with an aluminum substrate and a phosphor layer such as cesium iodide (CsI) and converts an X-ray to visible light, and a TFT panel, which includes a photoelectric transformation element for receiving the visible light from the scintillator panel and outputting an electric signal according to the intensity of the visible light. The scintillator (phosphor layer) is thrilled by depositing a cesium iodide (CsI) phosphor into a column-shaped monocrystal.

The X-ray photographing apparatus using the digital X-ray sensing module includes an X-ray source for generating an X-ray, a manipulation unit for receiving a user command for irradiating the generated X-ray on a subject to be examined, a digital X-ray sensing module for sensing the intensity of the X-ray transmitted through the subject to be examined and acquiring X-ray image data of the subject by converting the sensed intensity of the X-ray to an electric signal, and a management computer for initializing the digital X-ray sensing module before the X-ray is irradiated on the subject to be examined, and displaying the acquired image data on a display unit through image processing, or storing the processed image.

The X-ray source includes an X-ray tube for generating the X-ray, and a collimator for confirming an X-ray irradiation region of the subject to be examined. The X-ray tube has a cathode and an anode. When a high voltage is applied between the cathode and the anode, hot electrons are emitted from the cathode and strongly collide against the anode to generate the X-ray.

Meanwhile, most X-ray photographing apparatuses installed in medical centers using the film-type analog X-ray sensing modules. In the X-ray photographing apparatus using the film-type X-ray sensing module, a table for a subject to be examined is prepared, and the X-ray source is installed in a gantry such that it is spaced apart from the table by a predetermined. distance. A space for insertion of the film-type X-ray sensing module is prepared under the table.

If the analog sensing module of the related art X-ray photographing apparatus intends to be replaced with the digital X-ray sensing module, the whole X-ray photographing apparatus should be replaced. After everything except the X-ray source is removed from the conventional X-ray photographing apparatus, the digital X-ray sensing module may be installed. However, since the digital X-ray sensing module needs to be synchronized with the X-ray source, additional equipment for synchronization is necessarily required.

Therefore, enormous investments are required in order to replace the conventional X-ray photographing apparatus using the film-type X-ray sensing module with the digital X-ray photographing apparatus using the digital X-ray sensing module.

### SUMMARY OF THE INVENTION

Embodiments of the present invention overcome the above disadvantages and other disadvantages not described above. Also, the present invention is not required to overcome the disadvantages described above, and an embodiment of the present invention may not overcome any of the problems described above.

An aspect of the present invention is to provide a table type digital X-ray detector, which is capable of implementing a digital X-ray photographing apparatus through easy installation without replacing an existing X-ray photographing apparatus, a housing for the same, and an X-ray photographing apparatus using the same.

Other aspects, features and advantages of the present invention will become more apparent from the following description. In addition, objects and advantages of the present invention can be realized by means set forth in the claims.

According to an embodiment of the present invention, a table, type digital X-ray detector includes: a housing configured to be fixedly attachable to a support facing an X-ray source, at least a portion of the top surface of the housing being made of an X-ray transmittable material; and a digital X-ray sensing module embedded in the housing.

According to another embodiment of the present invention, a housing, in which a digital X-ray sensing module is fixedly installed and which is fixedly installed on a support facing an X-ray source, includes: a frame having a space for accommodating the digital X-ray sensing module: and a translucent or transparent cover made of an X-ray transmittable material.

According to another embodiment of the present invention, an X-ray photographing apparatus includes; an X-ray source; a support provided at a position facing the X-ray source; a. housing configured to be fixedly attachable to the support, at least a. portion of the top surface of the housing being made of an X-ray transmittable material; and a digital X-ray sensing module embedded in the housing.

### BRIEF DESCRIPTION OF THE DRAWINGS

The above and/or other aspects of the present invention will become apparent and more readily appreciated from the following description of embodiments, taken in conjunction with the accompanying drawings, of which:

FIG. 1 is a cross-sectional view illustrating an outer appearance of an X-ray photographing apparatus according to an embodiment of the present invention.

FIG. 2 illustrates an internal construction of a table type digital X-ray detector according to an embodiment of the present invention.

FIGS. 3A and 3B are exploded perspective views of a table type X-ray detector according to an embodiment of the present invention.

FIG. 3C is a partial sectional view illustrating a connection of a frame and a cover of FIGS. 3A and 3B according to an embodiment of the present invention.

### DETAILED DESCRIPTION OF THE INVENTION

Reference will now be made in detail to embodiments of the present invention, examples of which are illustrated in the accompanying drawings, wherein like reference numerals refer to like elements throughout. The embodiments are described below, in order to explain the present invention by referring to the figures.

Hereinafter, a table type digital X-ray detector and an X-ray photographing apparatus using the same according to embodiments of the present invention will be described in detail with reference to the accompanying drawings.

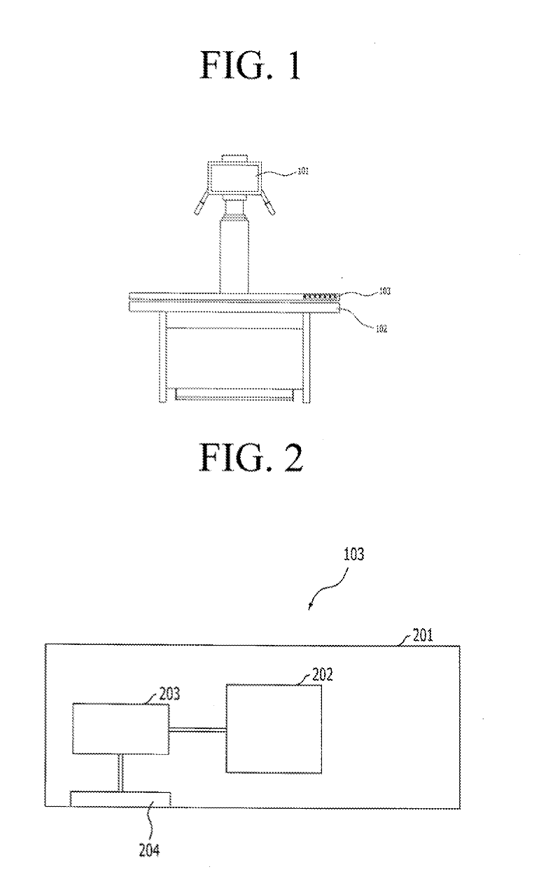

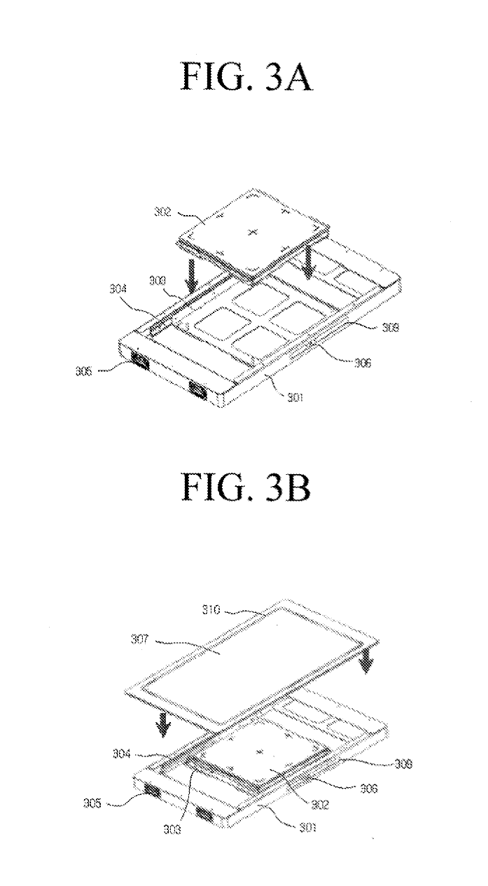

FIG. 1 is a cross-sectional view illustrating an outer appearance of an X-ray photographing apparatus according to an embodiment of the present invention, and FIG. 2 is a block diagram illustrating an internal construction of a table type digital X-ray detector according to an embodiment of the present invention.

An X-ray photographing apparatus according to an embodiment of the present invention may include a support 102, an X-ray source 101 disposed at a position facing the support 102 to irradiate an X-ray on a subject to be examined, and a table type digital X-ray detector 103 installed on the support 102.

The support 102 may be a table installed in a conventional film type X-ray photographing apparatus. That is, the conventionally used table may be used as the support 102 without extensive modification.

The table type digital X-ray detector 103 according to an embodiment of the present invention may be fixedly installed on the support 102 using an anti-sliding member so that it does not slide thereon. In other words, after the table type digital X-ray detector 103 is installed on the support 102, the anti-sliding member (not shown) is provided between the table type digital X-ray detector 103 and the support 102 in order to prevent the table type digital X-ray detector 103 from sliding on the support 102.

The anti-sliding member may be a rubber pad for preventing the sliding of the table type digital X-ray detector 103. In addition, the anti-sliding member may be an adhesive nonwoven fabric, a belt, a Velcro tape, a fixing bracket, a clamp, or element.

When the anti-sliding member is a rubber pad, the rubber pad may be attached to the top surface of the support 102 and/or the bottom surface of the housing of the table type digital X-ray detector 103. In addition, when the anti-sliding member is an adhesive nonwoven fabric, a belt, or a Velcro tape, the adhesive nonwoven fabric, the belt, or the Velcro tape may he provided on the top surface of the support 102 or the bottom surface of the housing of the table type digital X-ray detector 103. Meanwhile, if the anti-sliding member is a fastening structure such as a fixing bracket or a clamp, the bracket or the clamp may be fixed to the housing of the table-type digital X-ray detector 103. Since the anti-sliding member can be easily made using a general technique by those skilled in the art, specific embodiments thereof will be omitted.

As illustrated in FIG. 2, the table type digital X-ray detector 103 according to an embodiment of the present invention includes a housing 201, at least a portion of the top surface of which is made of an X-ray transmittable material, and a digital X-ray sensing module 202 installed inside the housing 201. The table type digital X-ray detector 103 according to an embodiment of the present invention may be manufactured in a size larger than or similar to that of the support 102, and thus, a height difference is not formed between the support 102 and the table type digital X-ray detector 103 when they are installed.

Although FIG. 2 illustrates that the peripheral circuit module 203 is installed inside the housing 201, it may also be installed outside the housing 201 and may be coupled to communicate with the digital X-ray sensing module 202. Referring to FIG. 2, in the case where the peripheral circuit module 203 is installed inside the housing 201, a port 204 coupled to an external power cable and a communication cable may be installed in the housing 201. In the case where the peripheral circuit module 203 is installed inside the housing 201, the peripheral circuit module 203 may be shielded by a noise shielding member (not shown) for preventing noise electromagnetic waves from being delivered to the digital X-ray sensing module 202.

Furthermore, the X-ray photographing apparatus according to an embodiment of the present invention may further include a photographing control unit for managing the control of the X-ray photographing, an irradiating control unit for managing the control of the X-ray source under the control of the photographing control unit, an input unit for receiving an X-ray photographing condition indicating a part to be photographed or an X-ray irradiation condition of the X-ray source, a display unit for displaying the X-ray photographing condition, and an irradiating switch for instructing an X-ray irradiation start.

In addition, the X-ray photographing apparatus according to an embodiment of the present invention may further include a driving unit and a driving control unit for allowing the X-ray source 101 to be movable in a vertical direction, and may further include a driving unit and a driving control unit for allowing the support 102 to be movable in a horizontal direction. Since the structure of the driving unit and the driving control unit is a known technique, a detailed description thereof will be omitted.

The housing 201 may be fixedly attachable to the support 102. The housing 201 may be made of an X-ray transmittable material having superior processability. For example, the housing 201 may be made of a metal such as aluminum, a wood such as MDF, a mixture of epoxy and nylon fiber, glass, plastic, or carbon fiber.

In the case where the housing 201 is made of wood, the housing 201 may be manufactured at a low cost because it is not necessary to manufacture a separate mold. However, there is a disadvantage in that the housing 201 is manufactured manually. In the case where the housing 201 is made of wood, a dampproof effect may be maximized by coating the outer surface of the housing 201.

In the case where the housing 201 is made of a synthetic resin, a manufacturing cost thereof may increase because it is necessary to manufacture a separate mold, but it may be advantageous for mass production.

The digital X-ray sensing module 202 installed inside the housing 201 may include a scintillator panel, an image sensor panel, and a protective cover. The scintillator panel may include a substrate and a phosphor layer formed on the substrate, and converts an X-ray to visible light. The image sensor panel may include a photoelectric transformation element for converting the visible light from the scintillator panel to an electric signal according, to the intensity of the visible light. The protective cover may protect the scintillator panel and the image sensor panel.

The digital X-ray sensing module 202 may be fixed inside the housing 201, and may be configured to adjust the position of the digital X-ray sensing module 202 according to the position of the X-ray source 101.

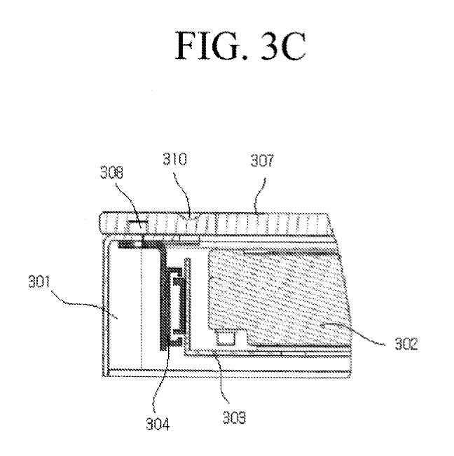

FIGS. 3A and 3B are exploded perspective views of a table type X-ray detector according to an embodiment of the present invention.

Referring to FIGS. 3A and 3B, the table type digital X-ray detector according to an embodiment of the present invention includes a frame 301, a digital X-ray sensing module 302, and a cover 307. The frame 301 may be installed such that the digital X-ray sensing module 302 is slidable. The cover 307 may be translucent or transparent and may he made of an X-ray transmittable material. A housing according to an embodiment of the present invention may include the frame 301 and the cover 307.

Referring to FIG. 3A, a sliding member 304 may he fixedly installed on both sidewalls in a long-length direction of the frame 301 such that the digital X-ray sensing module 302 is slidable. Since various commercial products of the sliding member 304 are available, detailed description about the structure of the sliding member 304 will he omitted.

The sliding member 304 may he fixedly connected to an accommodating part 303 for accommodating the digital X-ray sensing module 302. The accommodating part 303 may he similar in shape to the digital X-ray sensing module 302. Members for fixedly coupling the digital X-ray sensing module 302 to the accommodating part 303 may be provided.

Meanwhile, a manipulation part 306 for manipulating the sliding of the sliding member 304 may protrude from one side of the frame 301. A guide groove 309 for restricting a moving range of the manipulation part 306 may be formed. If a manipulation grip is pulled forward, a locking state of the manipulation part 306 may be released so to enable a slidable state. if the manipulation grip is returned to the original position, the manipulation part 306 returns to the locking state, i.e., a fixed state. Since various commercial products of the manipulation part 306 are also available, a detailed description thereof will be omitted.

A grip 305 for facilitating the movement of the housing may be provided on an outer side in a short-length direction, of the frame 301. The grip 305 may be configured so as not to protrude from the cross section of the frame 301.

Although manual adjustment of the position of the digital X-ray sensing module 302 has been described above, the position of the digital X-ray sensing module 302 can also be adjusted automatically using a driving motor or the like. Since specific methods for adjusting the position automatically can be easily implemented using techniques that have already been applied to a variety of electronic devices, a detailed description thereof will be omitted.

The digital X-ray sensing module 302 may be fixedly installed in the digital X-ray sensing module accommodating part 303 of the frame 301. A cross-shaped position indicator for indicating the position of the digital X-ray sensing module 302 may be formed on the digital X-ray sensing module 302.

Meanwhile, although not shown, a communication line for communicably connecting the digital X-ray sensing module 302 to an external device may be connected to the digital X-ray sensing module 302. Since the digital X-ray sensing module 302 may be movable, the communication line may be configured to be extensible according to the movement of the digital X-ray sensing module 302.

Referring to FIG. 3B, the cover 307 may be translucent or transparent and may be made of an X-ray transmittable material. A liquid flow prevention groove 310 for preventing the flow of liquid leaking from a subject to be examined, which is placed on the cover, may be formed along the upper edge of the cover 307.

FIG. 3C is a partial sectional view illustrates the connection of the frame 301 and the cover 307 in an embodiment of the present invention. Referring to FIG. 3C, a protrusion part 308 protruding from the cross section of the frame 301 may be formed on the frame 301. The protrusion part 308 may be configured using a screw for fixedly installing the sliding member 304 in the frame 301. A groove into which the protrusion part 308 is inserted may he formed on the bottom of the cover 307. Therefore, the cover 307 may he fixed to the frame 301 by placing the cover 307 on the frame 301 such that the groove formed on the bottom of the cover 307 is fit with the protrusion part 308 formed on the frame 301.

Meanwhile, the peripheral circuit module 203 may be electrically coupled to the digital X-ray sensing module 202 and may he embedded in the housing 201 or provided outside the housing 201. The peripheral circuit module 203 may include a power supply, an image sensor control circuit, and an external communication interface for communication with a computer as an external controller. Since heat radiating components may he included in the peripheral circuit module 203, a vent hole for heat sink (not shown) may be provided in the housing 201 in order to release heat generated by the peripheral circuit module 203.

Similarly, a peripheral circuit module (not shown) may be electrically coupled to the digital X-ray sensing module 302 and may be embedded in the frame 301 or provided outside the frame 301. The peripheral circuit module may include a power supply, an image sensor control circuit, and an external communication interface for communication with a computer as an external controller. Since heat radiating components may be included in the peripheral circuit module, a vent hole for heat sink (not shown) may be provided in the frame 301 in order to release heat generated by the peripheral circuit module.

The external communication interface may include cable/wireless communication modules, such as a cable LAN card, a wireless LAN card, a USB module, an infrared, module, a ZigBee module, and an optical communication module. Power may be directly supplied through a power cable or may be supplied from a rechargeable battery.

In addition, a wireless communication scheme using a radio frequency (RF) or an infrared light may be employed as a communication scheme for matching X-ray irradiation timing between the digital X-ray sensing module 202 or 302 and the X-ray source.

In an embodiment of the present invention, the X-ray irradiation timing between the digital X-ray sensing, module 202 or 302 and the X-ray source may be controlled by measuring an amount of current generated during the X-ray irradiation by using a high voltage cable and generating an image acquisition trigger signal to the digital X-ray sensing module 202 or 302. In other words, the high voltage cable may be installed on a path through which power is supplied to the X-ray source, and a current detector may be coupled to the high voltage cable. In this state, if power is supplied to the X-ray source in order to generate an X-ray, an induced current is generated in the high voltage cable, and the induced current may be detected by the current detector connected to the high voltage cable. In this manner, the point of time when the power is supplied to the X-ray source may be detected. The irradiation control unit may receive the current detection result from the current detector and transfer the image acquisition trigger signal to the digital X-ray sensing module.

In addition, the control unit for managing the overall control of the X-ray photographing apparatus may have a remote control function for initializing the digital X-ray sensing module When the digital X-ray sensing module fails.

Furthermore, a sound generation device may be embedded in the housing in order to help maintain psychological stability of an animal upon examination. A blood pressure meter, an electrocardiogram device, and a blood oxygen level meter may be embedded in the housing 201 or the frame 301 in order for additional inspection of a subject to be examined.

Moreover, the housing 201 may be configured to be assemblable or foldable using a hinge for easy long-distance movement. The foldable structure using the hinge may be implemented using a structure similar to that used for a travelling table or the like.

Meanwhile, a wheeled moving member for improving mobility may be installed on the bottom of the housing 201. In this case, the moving member may be formed in a “⊂” shape with one side of at least the long direction of the frame opened. Thus, the moving member may be moved in a direction opposite to the position where the X-ray source 101 is placed, and the table type digital X-ray detector 103 according to the present invention may be installed on the support.

In addition, a storage medium (e.g., a hard disk drive (HDD), a memory card, etc.) coupled to the peripheral module to store photographed data may be provided inside or outside the housing 201. Accordingly, when the X-ray photographing is completed, the image photographed by the digital X-ray detector may be stored in the storage medium by auto triggering. Then, the stored photographed image may be transferred to a management computer by cable or wirelessly.

Meanwhile, the housing 201 may include a socket into which the digital X-ray sensing module 202 can be inserted. Accordingly, the digital X-ray sensing module 202 may be configured so that it is inserted into the socket.

As described above, the existing film type X-ray photographing apparatus can be easily modified into a digital X-ray photographing apparatus by simply installing the film type X-ray sensing module on the table of the existing film type X-ray photographing apparatus.

Although embodiments of the present invention have been described herein, it should be understood that the foregoing embodiments and advantages are merely examples and are not to be construed as limiting the present invention or the scope of the claims. Numerous other modifications and embodiments can be devised by those skilled in the art that will fail within the spirit and scope of the principles of this disclosure, and the present teaching can also be readily applied to other types of apparatuses. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1-16. (canceled)

17. A digital X-ray detector comprising:
a housing configured to be positioned on a support facing an X-ray source, at least a portion of a top surface of the housing being made of an X-ray transmittable material; and
a digital X-ray sensing module embedded and configured to be movable within the housing wherein a position of the X-ray sensing module is adjusted according to a position of the X-ray source.

18. The digital X-ray detector of claim 17, further comprising an anti-sliding member configured to affix the housing to a top surface where the digital X-ray detector is disposed.

19. The digital X-ray detector of claim 18, further comprising a sliding member fixedly installed inside the housing to allow the digital X-ray sensing module to be movable within the housing.

20. The digital X-ray detector of claim 18, further comprising a liquid flow prevention member provided along an upper edge of the housing to prevent a flow of a liquid leaking from an examination object.

21. The digital X-ray detector of claim 17, wherein the housing includes a frame having a space configured to accommodate the digital X-ray sensing module within the housing.

22. The digital X-ray detector of claim 21, further comprising a liquid flow prevention member provided along an upper edge of the cover to prevent a flow of liquid leaking from an examination object.

23. The digital X-ray detector of claim 17, further comprising a manipulating part configured to manipulate the movement of the X-ray sensing module within the housing.

24. The digital X-ray detector of claim 22, further comprising a manipulating part configured to manipulate the movement of the X-ray sensing module within the housing wherein the manipulating part is protrude from the frame and the housing.

25. A housing, in which a digital X-ray sensing module is installed on a support facing an X-ray source, the housing comprising:
a frame configured to have a space for accommodating the digital X-ray sensing module and allowing the digital X-ray sensing module to move within the housing wherein a position of the X-ray sensing module is adjusted according to a position of the X-ray source; and
a translucent or transparent cover made of an X-ray transmittable material.

26. The housing of claim 25, further comprising a sliding member installed inside the frame to allow the digital X-ray sensing module to be slidable within the frame.

27. The housing of claim 25, further comprising a liquid flow prevention member provided along an upper edge of the cover to prevent a flow of a liquid leaking from an examination object.

28. An X-ray photographing apparatus comprising:
an X-ray source;
a support provided at a position facing the X-ray source;
a housing configured to be attachable to the support, at least a portion of a top surface of the housing being made of an X-ray transmittable material; and
a digital X-ray sensing module embedded and configured to be movable within the housing wherein a position of the X-ray sensing module is adjusted according to a position of the X-ray source.

29. The X-ray photographing apparatus of claim 28, further comprising an anti-sliding member configured to affix the housing to a top surface of the support.

30. The X-ray photographing apparatus of claim 28, further comprising a sliding member installed inside the housing to allow the digital X-ray sensing module to be slidable within the housing.

31. The X-ray photographing apparatus of claim 28, further comprising a liquid flow prevention member provided along an upper edge of the housing to prevent a flow of a liquid leaking from an examination object.

32. The X-ray photographing apparatus of claim 28, further comprising a timing controller configured to match an X-ray irradiation timing between the digital X-ray sensing module and the X-ray source.

33. The X-ray photographing apparatus of claim 28, wherein the timing controller comprises: a voltage supply detecting unit, coupled to a path through which a voltage is supplied to the X-ray source, configured to detect a point of time when the voltage is supplied to the X-ray source for X-ray generation; and a timing signal transmitting unit configured to transmit a trigger signal for image acquisition to the digital X-ray sensing module when a detection signal is input from the voltage supply detecting unit.

34. The X-ray photographing apparatus of claim 28, wherein the support is a table installed to an X-ray photographing apparatus configured to obtain X-ray images with X-ray films.

35. The X-ray photographing apparatus of claim 28, further comprising a manipulating part configured to manipulate the movement of the X-ray sensing module within the housing.

36. The X-ray photographing apparatus of claim 28, further comprising:
a frame having a space configured to accommodate the digital X-ray sensing module within the housing; and
a manipulating part configured to manipulate the movement of the X-ray sensing module within the housing wherein the manipulating part is protrude from the frame and the housing.
